(19) United States
(12) Patent Application Publication
(10) Pub. No.: US 2009/0001289 A1
(43) Pub. Date: Jan. 1, 2009

(54) Recording Medium

(76) Inventor: William S. Oakley, San Jose, CA (US)

Correspondence Address: TIPS GROUP;c/o Intellevate LLC, P. O. BOX 52050, Minneapolis, MN, 52050, US

(21) Appl. No.: 10/593,025

(22) PCT Filed: Mar. 14, 2005
(86) PCT No.: PCT/US2005/008035

§ 371 Date: Sep. 8, 2008

Related U.S. Application Data
(60) Provisional application No. 60/553,656, filed on Mar. 15, 2004.

(51) Int. Cl. A61N 5/00; B05D 1/36

(52) U.S. Cl. 250/492.3; 428/411.1; 427/402; 216/94

(57) ABSTRACT

A method and apparatus for a recording medium is described. In one embodiment, the invention is an apparatus. The apparatus includes a substrate. The apparatus also includes a phase change layer disposed on the substrate. The phase change layer has a first phase with a first secondary emission ratio and a second phase with a second secondary emission ratio.

### FIELD

The present invention, in some embodiments, generally relates to the field of media for data recording and more specifically to media for recording data using Carbon Nanotube electron emitters.

### BACKGROUND

Direct write electron beam writing has been used in lithography for wafer fabrication or mastering of optical discs. These systems typically use a 0.2 to 0.5 micron wide e-beam in a vacuum to expose a resist coated onto a substrate. In wafer fabrication the e-beam is typically modulated and scanned in a raster format to form an exposed pattern or image on a resist coated on a substrate. In optical disc mastering the resist coated disc is rotated beneath an e-beam. Neither of these applications relates to real-time data recording and neither is known to use Carbon Nanotubes.

Electron beam (e-beam) recording has traditionally been used to expose photographic film or resists for micro-lithographic purposes, both of which require post exposure processing. In a real time data record/playback system the sensitive layer cannot be processed after the exposure making these previous approaches unusable. Desirable writing processes for a real time memory application would be either archival or reversible depending on the application. While the e-beam recording of data marks is relatively straightforward, and generally results from either a chemical or heating process, reading of the written marks is far more difficult.

Thus, it may be useful to provide a writing and reading system that may be used in real-time. Furthermore, it may be useful to provide a medium for use in such a system.

### SUMMARY

A method and apparatus for a recording medium is described. In one embodiment, the invention is an apparatus. The apparatus includes a substrate. The apparatus also includes a phase change layer disposed on the substrate. The phase change layer has a first phase with a first secondary emission ratio and a second phase with a second secondary emission ratio.

In an alternate embodiment, the invention is a medium for use in a carbon nanotube drive. The medium includes a substrate. The medium also includes a first layer deposited on the substrate having a first secondary emission ratio. The medium further includes a second layer disposed on the first layer having a second secondary emission ratio. The first secondary emission ratio and the second secondary emission ratio differ by a factor detectable during secondary emission of electrons responsive to electrons from the carbon nanotube drive.

In another embodiment, the invention is an apparatus. The apparatus includes a substrate. The apparatus also includes a first layer deposited on the substrate having a first secondary emission ratio. The apparatus further includes a second layer disposed on the first layer having a second secondary emission ratio. The first secondary emission ratio and the second secondary emission ratio differ by at least a factor of 10.

In another alternate embodiment, the invention is a medium for use in a carbon nanotube drive. The medium includes a substrate. The medium also includes a phase change layer disposed on the substrate. The phase change layer has a first phase with a first secondary emission ratio and a second phase with a second secondary emission ratio.

In another embodiment, the invention is a method. The method includes receiving electrons at a spot of a phase change material having a first phase and a second phase. The first phase has associated therewith a first secondary emission ratio, and the second phase has associated therewith a second secondary emission ratio. The method also includes absorbing the electrons within a portion of the phase change material. The portion is aligned with the spot. The portion is in the first phase prior to absorbing the electrons. The method further includes changing the portion of the phase change material to the second phase responsive to absorbing the electrons.

In yet another alternate embodiment, the invention is a method. The method includes receiving a substrate. The method further includes depositing on the substrate a first layer of material having a first secondary emission ratio.

In still another embodiment, the invention is a disk drive. The disk drive includes an enclosed medium. The enclosed medium includes a substrate. The enclosed medium also includes a first layer deposited on the substrate having a first secondary emission ratio. The enclosed medium further includes a second layer disposed on the first layer having a second secondary emission ratio. The disk drive also includes an actuator positioned to move within the disk drive in proximity to the medium. The disk drive further includes a read/write head coupled to the actuator. The head includes a substrate. The head also includes a carbon nanotube mounted on the substrate. The head further includes an extraction electrode mounted in proximity to a tip of the carbon nanotube.

In yet another embodiment, the invention is a method. The method includes receiving electrons at a spot of a first layer of a medium. The first layer is disposed above a second layer. The first layer has a first secondary emission ratio and the second layer has a second secondary emission ratio. The first secondary emission ratio differs from the second secondary emission ratio. The method also includes removing a portion of the first layer responsive to receiving the electrons, with the portion aligned with the spot.

In still another embodiment, the invention is a method. The method includes projecting electrons from a carbon nanotube at a spot of a first layer of a medium. The first layer is disposed above a second layer. The first layer has a first secondary emission ratio and the second layer has a second secondary emission ratio. The first secondary emission ratio differs from the second secondary emission ratio. The number and energy of electrons projected is based on an expected amount of energy to remove a portion of the first layer, with the portion aligned with the spot. The method further includes removing the portion of the first layer responsive to receiving the electrons.

In another embodiment, the invention is a method. The method includes projecting electrons from a carbon nanotube at a spot of a phase change material. The phase change material has a first phase and a second phase. The first phase has associated therewith a first secondary emission ratio. The second phase has associated therewith a second secondary emission ratio. The method also includes absorbing the electrons within a portion of the phase change material. The portion is aligned with the spot. The portion is in the first phase prior to absorbing the electrons. The method further includes changing the portion of the phase change material from the first phase to the second phase responsive to absorbing the electrons.

In yet another embodiment, the invention is a disk drive. The disk drive includes an enclosed medium. The enclosed medium includes a substrate. The enclosed medium also includes a phase change layer disposed on the substrate having a first phase and a second phase. The first phase has a first secondary emission ratio and the second phase has a second secondary emission ratio. The disk drive also includes an actuator positioned to move within the disk drive in proximity to the medium. The disk drive further includes a read/write head coupled to the actuator. The head includes a substrate. The head also includes a carbon nanotube mounted on the substrate. The head further includes an extraction electrode mounted in proximity to a tip of the carbon nanotube.

In still another alternate embodiment, the invention is an apparatus. The apparatus includes a first means for emitting secondary electrons at a first rate. The apparatus also includes a second means for emitting secondary electrons at a second rate. The apparatus further includes a support means for supporting the first means and the second means.

### BRIEF DESCRIPTION OF THE DRAWINGS

The present invention is illustrated in various embodiments by way of example and not limitation in the accompanying figures, in which like numbers represent like or similar components.

FIG. 1 illustrates a relationship between distance from a beam source and corresponding beam width.

FIG. 2a illustrates an embodiment of an apparatus useful in producing an electron beam using a Carbon Nanotube.

FIG. 2b illustrates an alternate embodiment of an apparatus useful in producing an electron beam using a Carbon Nanotube.

FIG. 3 illustrates another alternate embodiment of an apparatus useful in producing an electron beam using a Carbon Nanotube.

FIG. 4 illustrates an embodiment of a method of using a storage device using a Carbon Nanotube.

FIG. 5 illustrates an alternate embodiment of a method of using a storage device using a Carbon Nanotube.

FIG. 6a illustrates an embodiment of an apparatus that may be used for recording on media

FIG. 6b illustrates an alternate embodiment of an apparatus that may be used for recording on media.

FIG. 7 illustrates yet another alternate embodiment of an apparatus useful in producing an electron beam using a Carbon Nanotube.

FIG. 8 illustrates an embodiment of a method of making a storage device using a Carbon Nanotube.

FIG. 9 illustrates an embodiment of a method of using a storage device including a Carbon Nanotube.

FIG. 10 illustrates an embodiment of a read-only medium for use in a storage device.

FIG. 11 illustrates an alternate embodiment of a read-only medium for use in a storage device.

FIG. 12 illustrates an exemplary signal received when reading a medium in a storage device.

FIG. 13 illustrates an embodiment of a phase change medium for use in a storage device.

FIG. 14 illustrates a method of making a phase change medium for use in a storage device.

FIG. 15 illustrates a method of writing a phase change medium for use in a storage device.

FIG. 16 illustrates a method of reading a phase change medium for use in a storage device.

FIG. 17 illustrates an embodiment of a write-once medium for use in a storage device.

FIG. 18 illustrates a method of making a write-once medium for use in a storage device.

FIG. 19 illustrates a method of writing a write-once medium for use in a storage device.

FIG. 20 illustrates a method of reading a destructive write medium for use in a storage device.

### DETAILED DESCRIPTION

A method and apparatus for a recording medium is described. In the following description, for purposes of explanation, numerous specific details are set forth in order to provide a thorough understanding of the invention. It will be apparent, however, to one skilled in the art that the invention can be practiced without these specific details. In other instances, structures and devices are shown in block diagram form in order to avoid obscuring the invention.

Reference in the specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment, nor are separate or alternative embodiments mutually exclusive of other embodiments.

In one embodiment, the invention is an apparatus. The apparatus includes a substrate. The apparatus also includes a phase change layer disposed on the substrate. The phase change layer has a first phase with a first secondary emission ratio and a second phase with a second secondary emission ratio.

In an alternate embodiment, the invention is a medium for use in a carbon nanotube drive. The medium includes a substrate. The medium also includes a first layer deposited on the substrate having a first secondary emission ratio. The medium further includes a second layer disposed on the first layer having a second secondary emission ratio. The first secondary emission ratio and the second secondary emission ratio differ by a factor detectable during secondary emission of electrons responsive to electrons from the carbon nanotube drive.

In another embodiment, the invention is an apparatus. The apparatus includes a substrate. The apparatus also includes a first layer deposited on the substrate having a first secondary emission ratio. The apparatus further includes a second layer disposed on the first layer having a second secondary emission ratio. The first secondary emission ratio and the second secondary emission ratio differ by at least a factor of 10.

In another alternate embodiment, the invention is a medium for use in a carbon nanotube drive. The medium includes a substrate. The medium also includes a phase change layer disposed on the substrate. The phase change layer has a first phase with a first secondary emission ratio and a second phase with a second secondary emission ratio.

In another embodiment, the invention is a method. The method includes receiving electrons at a spot of a phase change material having a first phase and a second phase. The first phase has associated therewith a first secondary emission ratio, and the second phase has associated therewith a second secondary emission ratio. The method also includes absorbing the electrons within a portion of the phase change material. The portion is aligned with the spot. The portion is in the first phase prior to absorbing the electrons. The method further includes changing the portion of the phase change material to the second phase responsive to absorbing the electrons.

In yet another alternate embodiment, the invention is a method. The method includes receiving a substrate. The method further includes depositing on the substrate a first layer of material having a first secondary emission ratio.

In still another embodiment, the invention is a disk drive. The disk drive includes an enclosed medium. The enclosed medium includes a substrate. The enclosed medium also includes a first layer deposited on the substrate having a first secondary emission ratio. The enclosed medium further includes a second layer disposed on the first layer having a second secondary emission ratio. The disk drive also includes an actuator positioned to move within the disk drive in proximity to the medium. The disk drive further includes a read/write head coupled to the actuator. The head includes a substrate. The head also includes a carbon nanotube mounted on the substrate. The head further includes an extraction electrode mounted in proximity to a tip of the carbon nanotube.

In yet another embodiment, the invention is a method. The method includes receiving electrons at a spot of a first layer of a medium. The first layer is disposed above a second layer. The first layer has a first secondary emission ratio and the second layer has a second secondary emission ratio. The first secondary emission ratio differs from the second secondary emission ratio. The method also includes removing a portion of the first layer responsive to receiving the electrons, with the portion aligned with the spot.

In still another embodiment, the invention is a method. The method includes projecting electrons from a carbon nanotube at a spot of a first layer of a medium. The first layer is disposed above a second layer. The first layer has a first secondary emission ratio and the second layer has a second secondary emission ratio. The first secondary emission ratio differs from the second secondary emission ratio. The number and energy of electrons projected is based on an expected amount of energy to remove a portion of the first layer, with the portion aligned with the spot. The method further includes removing the portion of the first layer responsive to receiving the electrons.

In another embodiment, the invention is a method. The method includes projecting electrons from a carbon nanotube at a spot of a phase change material. The phase change material has a first phase and a second phase. The first phase has associated therewith a first secondary emission ratio. The second phase has associated therewith a second secondary emission ratio. The method also includes absorbing the electrons within a portion of the phase change material. The portion is aligned with the spot. The portion is in the first phase prior to absorbing the electrons. The method further includes changing the portion of the phase change material from the first phase to the second phase responsive to absorbing the electrons.

In yet another embodiment, the invention is a disk drive. The disk drive includes an enclosed medium. The enclosed medium includes a substrate. The enclosed medium also includes a phase change layer disposed on the substrate having a first phase and a second phase. The first phase has a first secondary emission ratio and the second phase has a second secondary emission ratio. The disk drive also includes an actuator positioned to move within the disk drive in proximity to the medium. The disk drive further includes a read/write head coupled to the actuator. The head includes a substrate. The head also includes a carbon nanotube mounted on the substrate. The head further includes an extraction electrode mounted in proximity to a tip of the carbon nanotube.

In still another alternate embodiment, the invention is an apparatus. The apparatus includes a first means for emitting secondary electrons at a first rate. The apparatus also includes a second means for emitting secondary electrons at a second rate. The apparatus further includes a support means for supporting the first means and the second means.

The present invention relates generally to using a Carbon Nanotube as a source for an electron beam suitable for real-time writing of data to a storage medium. Carbon Nanotube electron emitters are used as electron sources for recording data marks onto various recording media B-beam writing has generally not been used directly for real-time data storage. Similarly, carbon nanotubes have not previously been used as electron sources for e-beam recording, or for resist exposure on wafer substrates or discs.

A data storage system may use the electron beam emitted from a single carbon nanotube impinging onto a sensitive recording media and thereby recording a mark corresponding to an input data signal that controls the nanotube output to the media. The input signal data may be converted to either analog or digital modulation of the emitted electron beam either at the nanotube or otherwise but before reaching the recording media. The carbon nanotube emitter may be in close proximity to the recording media such that no electron lens system is required to form the desired recorded mark size. Alternatively, an electron lens system may be interposed between the emitter and the recording media such that the emitted beam is focused to write a recorded mark on the media.

The emitter and media may both be located in a vacuum enclosure. Alternatively, the emitter may be located in a vacuum enclosure and the recording media may reside in a gaseous domain either at atmospheric or reduced pressure. Similarly, the emitter may be located in a vacuum enclosure and the recording media may reside in a liquid domain. The recording media may be in the form of a rotating disc or the form of a long translating tape. The recording media may either be of a reversible (or erasable nature) or of a permanent archival nature. Similarly, the recording media may be sensitive to either radiation by electrons or thermally sensitive and undergo a change so as to record a data mark. Alternatively, the recording media may be a material that undergoes a phase change when subjected to the electron beam energy.

The recording media may be either preformatted or unformatted media. The recording media may either use or not use a protective layer over the sensitive layer with either a thin or substantially thicker protective layer. The media may have an electron permeable upper layer.

The e-beam may pass through an electron permeable membrane between the nanotube emitter and the recording media. The electron permeable membrane may be placed so as to enable the nanotube emitter and an electron lens to operate in a vacuum environment while interacting with recording media which is not in vacuum.

Systems may use a multiplicity of similar nanotube emitters arranged in a specific pattern to form a similar pattern of beams impinging on the recording media, some or all of which produce recorded marks. The array pattern of nanotube emitters can be distributed in one, two, or three dimensions as appropriate for the specific system design. Bach nanotube in the array may be individually modulated by a data or formatting signal. Some selected nanotube emitters in the array of multiple emitters may be used for tracking formatting and/or data marks. The nanotube emitter assembly may be mounted on a moving element to enable accurate tracking of recorded formatting marks and recorded data marks via a servo system that drives the moving assembly. The emitter assembly may contain a means of beam deflection to move the recording/reading beam array in a transverse manner to enable precision tracking of the recorded data and format marks and where such deflection means is either electrostatic or electromagnetic in nature.

The emission by carbon nanotubes of electron beams of significant power, nearly collimated, and with a small spread in electron velocities make these devices useful as sources for advanced electron beam data recorders. The recorder design can encompass either a single nanotube or an array of nanotubes in a preferred pattern and can enable either rotating disc or tape recorder designs of very high capacity and data rate. The basic design approaches can be defined by two parameters. One is wherein the carbon nanotube is either located in close proximity to the recording surface, or is located further from the recording surface necessitating an electron lens to refocus the beam to a desirably small size at the recording media sensitive surface. The second is whether the recording media is either located in the same vacuum enclosure as the emitting e-beam source, or a means is provided whereby the recording media can be located in another region, such as one containing gasses at atmospheric or reduced pressure for example. In this circumstance an electron permeable membrane is useful to maintain the emitting nanotube in a vacuum.

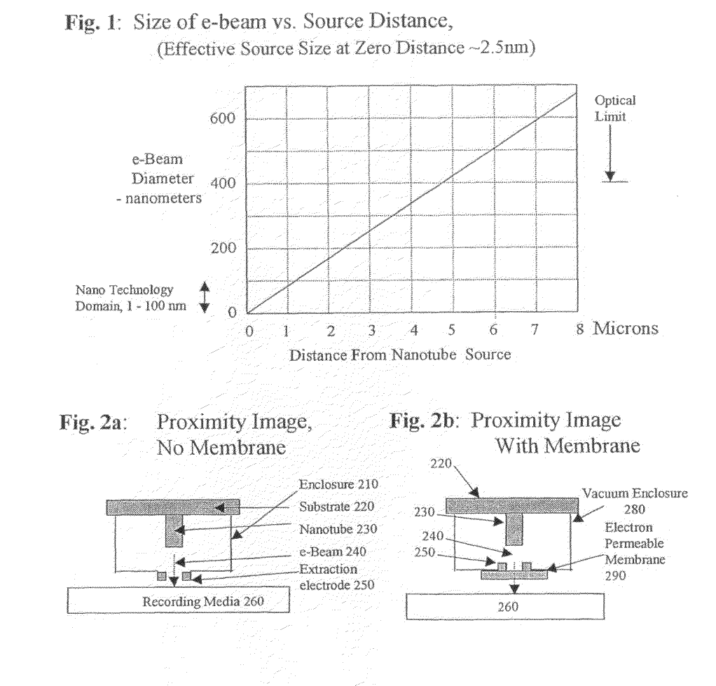

In one embodiment, a recorder system allows the media to be removed and replaced by another media volume or removed and placed in another similar recorder or reader. This involves the carbon nanotube emitting source (CNTES), or array of such sources, either located very close to the recording media or located in a vacuum enclosure separated from the media by an electron permeable membrane. In a close proximity design, the electron permeable membrane may be located between the CNTES and the media and preferably as close to the media as is mechanically possible. For disc systems this may be on the order of a micron, but for tape systems may be substantially greater due to the dynamics of tape motion. As seen from FIG. 1, the electron beam emanating from a typical carbon nanotube appears as though from a very small source, somewhat smaller than the diameter of the nanotube, and slowly expands with increasing distance from the source. The e-beam diameter is about 100 nanometers at a distance of 1.2 microns from the CNTES, easily compatible with flying a read/write head at this height above a media surface. Typical head flying heights in (sealed) hard disc drives are small fractions of a micron, compatible with beam diameters of 10 nanometers of less. In high-speed tape systems the tape flying height is typically about 1 micron above the magnetic head or above the tape support member for optical tape drives. The flying head concept for proximity located CNTES without and with an electron permeable membrane are shown in FIGS. 2a and 2b.

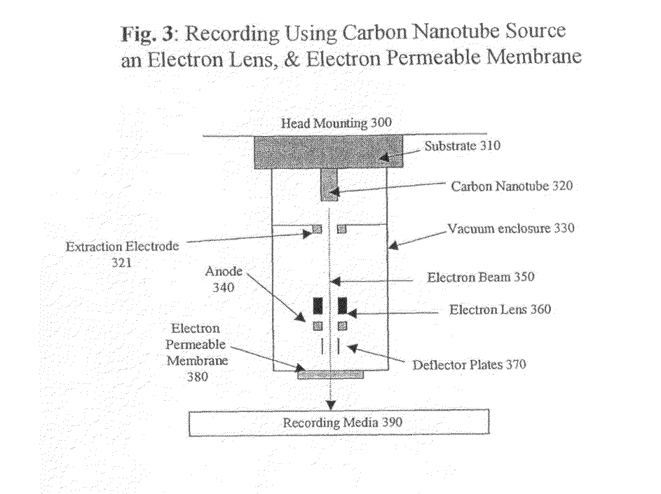

In systems employing an electron lens to focus the beam onto the media the electron permeable membrane may be located as close to the media as practical, and lies between the electron lens and the media as shown in FIG. 3. Separation distances between the media surface and the membrane of from a few up to the order of 100 microns may be feasible.

In designs where an electron permeable membrane is employed the electron accelerating voltage can be substantially increased to improve membrane penetration with voltages in the 1 kv to 3 kV (one to three kilovolt) range being preferred. This increased voltage also increases the power of the electron beam, for example a 200 nanoamp current at 3 kV provides a beam power of 0.6 milliwatts. In a beam diameter of 50 nm for example, this is a power density of about 2×10⁶ Watts per square centimeter. To avoid damage to the membrane it may be advantageous to locate an electron lens inside the vacuum region and configure the beam geometry so the beam diameter at the membrane is relatively large, thus minimizing the power density. This lens should bring the beam to focus outside of the vacuum region and at the sensitive layer in the recording media.

A thin dielectric membrane of heat resisting material possessing high mechanical strength may be useful, such as a thin sheet of silicon nitride, boron carbide, silicon carbide, or similar material. The thin membrane, typically several tens of nanometers thick, may be attached to the container or enclosure and cover an aperture or window through which the beam can pass. In one embodiment, the aperture in the enclosure is elongated enabling the beam to be deflected for tracking purposes if desired. A minimum sized aperture may be useful to minimize gas leakage into the vacuum enclosure and also to minimize the force on the membrane due to the difference in pressures inside and outside the container. For example, a membrane covering an aperture of 0.1×0.4 mm has to withstand a nominal force of approximately 0.5 grams at one atmosphere pressure.

In all embodiments, a means of tracking format marks or data tracks may be useful. Dynamic tracking of these marks by electrostatic or magnetic deflection of the e-beam, or array of beams, may easily be achieved but is only viable for small off-axis deflections. This small deflection is typically adequate to compensate for track run-out on a given track, but is typically not sufficient to provide for cross track access. For this reason the CNTES and electron lens, if any, and electron permeable membrane if any, may be located on a movable read/write head that provides cross track motion. Both cross track seek and track following may be achieved by servo control systems using feedback.

Turning to further details of the figures, FIG. 1 illustrates a relationship between distance from a beam source and corresponding beam width. The beam width increases roughly linearly with distance from the source. Optically visible light has a limit of about 400 nm, and nanotechnology typically operates in the 1-100 nm area. At a distance of about 1.2 microns from the source, a 100 nm wide beam may be expected as described above.

FIG. 2a illustrates an embodiment of an apparatus useful in producing an electron beam using a Carbon Nanotube. Such a beam may be expected to have a width predictable based on the graph of FIG. 1. An enclosure 210 may be part of a head assembly. Within the enclosure 210, typically affixed to one or more surfaces of the enclosure 210 is substrate 220. Mounted on substrate 220 is nanotube 230, such as a carbon nanotube. Emitted from nanotube 230 is electron beam 240, which may be used to record a mark on recording media 260. Recording media 260 may be a disk or tape having a surface or layer that is sensitive to electrons of electron beam 240. The extraction electrode 250 helps cause the electron beam 240 to be emitted from nanotube 230, such as by creating a potential (voltage) difference between the nanotube 230 and the electrode 250 (such as by using an outside source of voltage differential coupled to both of nanotube 230 and electrode 250).

FIG. 2b illustrates an alternate embodiment of an apparatus useful in producing an electron beam using a Carbon Nanotube. Unlike FIG. 2a, this embodiment uses a vacuum enclosure 280 on which is mounted substrate 220. An electron permeable membrane 290 is used to seal the opening through which the electron beam 240 passes, thereby allowing for a sealed vacuum environment for the nanotube 230 and a non-vacuum environment for media 260.

FIG. 3 illustrates another alternate embodiment of an apparatus useful in producing an electron beam using a Carbon Nanotube. Head mounting 300 is the head assembly in which this is incorporated. Vacuum enclosure 330 is mounted on mounting 300. Substrate 310 is mounted within enclosure 330, such as by mounting to the end of enclosure 330. Nanotube 320 is affixed to substrate 310, allowing for electrical and mechanical connections. Anode 340 is mounted within enclosure 330, allowing for generation of a potential difference between the anode 340 and extraction electrode 321. Electron beam 350 is produced in response to a potential difference between the carbon nanotube 320 and the extraction electrode 321, is accelerated by the potential difference between the extraction electrode 321 and the anode 340, and may be focused by electron lens 360, and/or deflected by deflector plates 370, before passing through electron permeable membrane 380 and out of enclosure 330 to make a mark on recording medium (or media) 390.

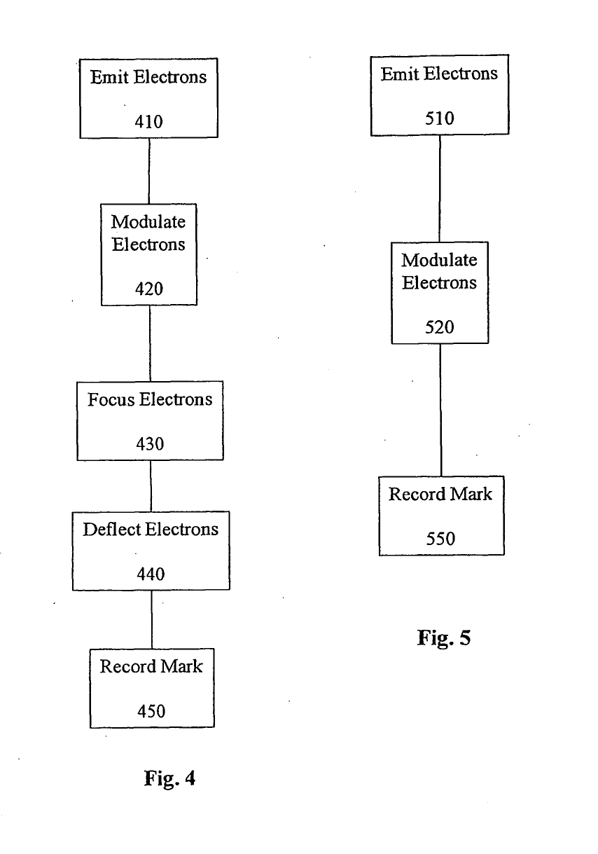

Various methods of using storage devices may be suitable depending on design and operating conditions. FIG. 4 illustrates an embodiment of a method of using a storage device using a Carbon Nanotube. At block 410, electrons are emitted, such as in response to a potential difference between a nanotube and the extraction electrode. At block 420, the electron beam intensity is modulated, such as by varying the extraction electrode voltage (not shown in the illustrations). Such a modulation may be used to either selectively block the electrons, or modulate the beam intensity. The modulator 420 may also include a means of beam deflection to selectively deflect the electrons, in response to a deflection signal. At block 430, the electrons of the beam formed by the emitted electrons are focused. At block 440, the electrons are deflected to correct for deviations from a path expected during design of the system. At block 450, the electrons record a mark on a recording medium.

FIG. 5 illustrates an alternate embodiment of a method of using a storage device using a Carbon Nanotube. At block 510, electrons are emitted, such as from a carbon nanotube. At block 520, the electrons are modulated based on a modulation signal. At block 550, the electrons record a mark on a recording medium. In the processes of both FIGS. 4 and 5, blocks may be reordered, rearranged, or combined, depending on design constraints and preferences, within the spirit and scope of the present invention. For example, modulation may relate to whether electrons are emitted or not, rather than whether electrons already emitted ultimately reach a recording medium.

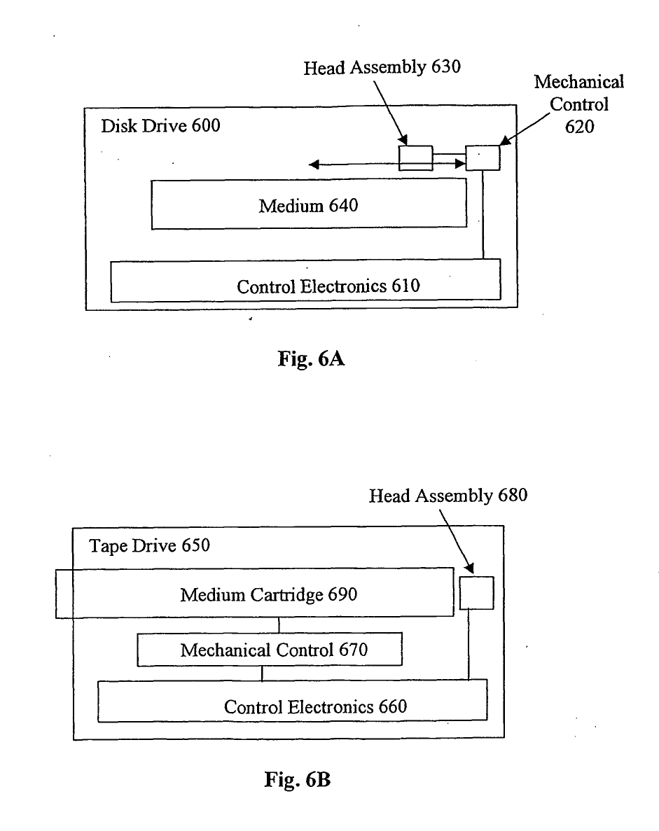

The embodiments of methods and apparatuses previously described may be used in various systems. FIG. 6a illustrates an embodiment of an apparatus that may be used for recording on media. Disk drive 600 includes control electronics 610, mechanical control 620, head assembly 630, and medium 640. Note that multiple similar or identical components may be included, such as a set or media 640 and corresponding set of head assemblies 630 and mechanical controls 620. In one embodiment, an interface with control electronics 610 allows for communication with components attached to or coupled to disk drive 600. Control electronics 610 controls mechanical control 620. Mechanical control 620 actuates head assembly 630, causing the head assembly to move in a range between a center and edge limit. Media 640 is a disk which may be spun on a spindle (not shown) for example, such that the head assembly may effectively move to any location on media 640 and either record or read data Head assembly, in some embodiments, is implemented using embodiments such as those illustrated in FIGS. 2a, 2b and 3 for example.

FIG. 6b illustrates an alternate embodiment of an apparatus that may be used for recording on media. Tape drive 650 includes control electronics 660, mechanical control 670, head assembly 680, and space for medium cartridge 690. Medium cartridge 690 is a self-contained tape cartridge allowing for access to the tape near the location head assembly 680 and manipulation of the tape (on spools for example) by mechanical control 670. Head assembly 680 and mechanical control 670 operate responsive to control electronics 660. Control electronics 660 may interface with external components to receive and send data.

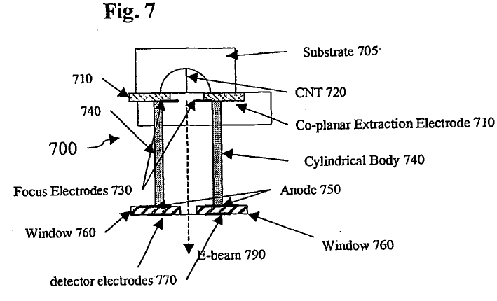

As described above, FIG. 3 illustrates an embodiment of a very small read/write head using a CNT as an electron emitter. In alternate embodiments, several of these components can be combined to facilitate device fabrication. An alternate embodiment of a read write head 700 may be as shown in FIG. 7, where an extraction electrode 710 with a central aperture is placed near the CNT 720 and is maintained at ground electrical potential. The CNT 720 tip may be located a micron or two (for example) from the extraction electrode 710 and is preferably precisely centered on the electrode aperture. The CNT 720 may be maintained at a few volts negative relative to the extraction electrode 710. Modulation of the emitted e-beam 790 may then be achieved by reducing the extraction electrode 710 voltage to at or below the CNT 720 voltage, thus gating the e-beam 790 current. With the appropriate drive circuits, modulation at a rate of hundreds of gigahertz or higher may be achievable.

As illustrated in this embodiment, substrate 705 includes a cavity that includes CNT 720. Extraction electrodes 710 are preferably formed on an approximately planar surface of substrate 705 in one embodiment. Focus electrodes 730 may then be formed along with extraction electrodes 710 or in a nearby location, allowing for focus and/or deflection of emitted e-beam 790. Cylindrical dielectric body 740 may surround the aperture through extraction electrodes 710 and/or focus electrodes 730. Closing cylindrical dielectric body 740 may be window or cover 760, which preferably is electron permeable but relatively vacuum-proof (allows for maintenance of an evacuated environment within body 740). Within body 740 on or near window 760 are formed anode(s) 750. Outside of window 760 are formed detectors (detector electrodes for example) 770.

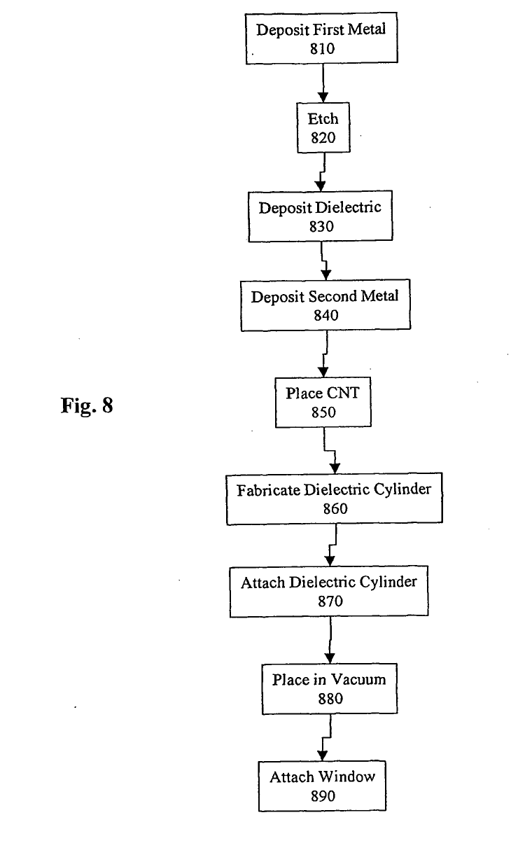

The embodiment of FIG. 7 may be produced using a method such as the method illustrated in FIG. 8 for example. In one embodiment, the fabrication of the extraction electrode is by metal deposition onto a wafer substrate that is then etched away to leave the electrode patterned as a disc with a central aperture and connecting traces. In such an embodiment, a dielectric layer may then be deposited onto the extraction electrode to insulate the electrode from further depositions. Additionally, in such an embodiment, a second metallic deposition of an annular electrode segmented into quadrants may be placed onto the dielectric to form a focus electrode. These electrode quadrants may form an annular ring that lies outside the extraction electrode disc and is nearly co-planar. In such an embodiment, adjusting the voltage of all of the electrode segments in unison may allow for focusing the e-beam. Placing slightly different voltages on the appropriate electrode quadrants may enable the e-beam to be deflected away from the beam (z) axis in either or both x and y directions. Thus, the extraction electrode, the focus electrode and the deflection electrodes may be fabricated in a single structure, thereby eliminating the need for separate electrode structures within the read/write head body (enclosure).

In such an embodiment, the entire microscale read/write head may be fabricated in three pieces prior to assembly. First the CNT emitter with the extraction electrode and focus/deflection electrodes is fabricated. Next, a body of dielectric is fabricated and attached to the emitter assembly. The combined assembly is then placed in a vacuum of approximately 10⁻⁸ torr (for example) and the window is attached, sealing the CNT head assembly. The window consists of a silicon membrane typically 30 nanometers thick (for example) mounted in a carrier with a circular anode on the inner surface (for electron acceleration) and a read detector electrode on the outer surface. Both anode and detector electrodes preferably have connections or couplings to electrical circuits.

Thus, FIG. 8 may also be described as a process including a set of modules. At module 810, the first metal deposition (initial electrode) occurs. At module 820, the first metal is etched (initial electrode etch). At module 830, the dielectric is deposited. At module 840, the second metal is deposited (annular electrode deposition). At module 850, the CNT emitter is placed within the central aperture. At module 860, the dielectric enclosure or body is fabricated. At module 870, the dielectric body is attached to the CNT emitter structure. At module 880, the combined structure is placed in a vacuum. At module 890, the window with electrodes is attached.

In one embodiment, the read write data storage system may be implemented with a higher beam power causing a change in the recording media that is then detectable by a lower power read beam by either secondary emission of electrons or by e-beam fluorescence.

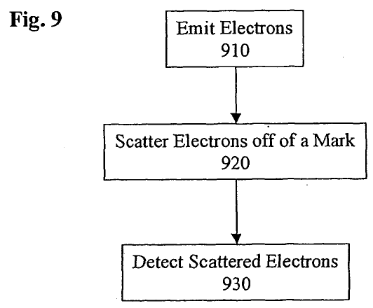

FIG. 9 illustrates an embodiment of a method of using a storage device including a Carbon Nanotube. Reading marks made by a carbon nanotube is a necessary operation to use the carbon nanotube for data storage. At module 910, an e-beam is emitted. At module 920, the e-beam illuminates a mark on a storage medium. Note that the e-beam may need to be deflected and/or focused, too. At module 930, electrons from the mark, such as secondary electrons, are detected by a detector. Such a detector may use either secondary emission of electrons or alternately detect photons emitted from the mark by e-beam fluorescence, for example.

### Alternate Design Material

The Carbon Nano Tube is preferred as a nanoscale electron emitter due to several inherent characteristics arising from its carbon composition. Another option would be to use a silicon nano tip in a similar structure where the radius of the tip is similar to the radius of the nanotube, e.g. a few nanometers, providing similar source sizes. However, in similar designs the electron current emission capability of a CNT is at least ten times that of a silicon tip for several reasons. Firstly the CNT is essentially a single molecule of carbon and has greater electrical conductivity than silicon. This permits greater current through the CNT than the silicon tip for any given degree of resistive heating, which is a typical limiting factor in performance. Secondly, the CNT has a higher melting temperature than silicon so it is better able to withstand a given temperature without detrimental mechanical effects. Thirdly the CNT is a long cylindrically shaped structure that places the tip far from the base whereas the silicon tip is essentially pyramidal. This allows the CNT to effectively provide a greater ratio of height/radius, a ratio that relates directly to the electric field enhancement factor of the structure, enabling the same emission field at a lower applied voltage for the CNT structure compared to a silicon tip.

All these factors notwithstanding, if a particular gated emitter design requires a lower emission current; for example if due to a smaller recorded spot size only 1/25 of the CNT maximum current is required, then the silicon tip may prove viable. Typical material parameters indicate a silicon tip emitter may be feasible at spot sizes smaller than 10 nanometers diameter. Accordingly, CNTs 720 and 320 may be replaced with silicon tip emitters in some embodiments. While this may require other modifications due to various engineering constraints, undue experimentation should not be necessary to make such a replacement.

Media

Media for use in a CNT read/write drive may take many forms. For example, such media may be write-once, read-only (pre-written), or rewritable. Such media may include materials which vaporize or melt, depending on materials availability and other design considerations.

As mentioned, the e-beam recording of data marks is relatively straightforward, and generally results from either a chemical or heating process, but reading of the written marks is far more difficult. One possible means of reading is by comparing the difference in Secondary Emission (SE) characteristics of written media marks with that of unwritten media. The SE ratio (δ) is the number of secondary electrons emitted from a material when impacted by a primary electron. Several approaches are evident for the recording and reading of data.

In one embodiment, a media substrate coated with a single thin layer of material that is heated and vaporized by the e-beam leaving a bare substrate with a different SE rate than the coating is used. The selective removal of material provides a means of permanently recording data. In this approach the write beam characteristics should be matched to the sensitive material vaporization energy. The write e-beam should be of high current and of just sufficient voltage to be just fully absorbed within, but not penetrate significantly beyond, the sensitive layer. The read beam should be of less current and of lower total power so as not to overwrite the data. The read voltage should be selected to maximize the SE return from the material with the highest SE ratio, either the top layer or the substrate. This is generally an irreversible process.

In an alternate embodiment, a single coating with secondary emission properties altered by exposure to an e-beam of sufficient intensity so as to cause a phase change (PC) in the material, but without material removal is used. The two phase states of the PC material provide a means of data recording due to the differing SE ratios of the phases. This may be a reversible process.

In another alternate embodiment, a double layer coating on a substrate is used, the first layer is vaporized to expose the second layer composed of a material with a significantly different secondary emission output. This is essentially identical to the first approach but the lower layer is not the substrate, allowing greater design freedom of choice of material. This is generally an irreversible process.

In still another alternate embodiment, a three layer structure is used, with the first layer selected for one SE rate, the second layer for an appropriate vaporization temperature and the third layer for a second SE ratio. The combined thickness of the first two layers should be just sufficient to completely absorb the write beam. Vaporizing the second layer will also carry away the first layer, exposing the third layer for reading. The SE ratio difference between the first and second layers provides the recorded data signal. This is generally an irreversible process.

In yet another alternate embodiment, a multi layer structure is used, where the beam energy is selectable to remove successive layers of different SE ratios to provide multi level data encoding.

In still another alternate embodiment, the media can also be structured as Read Only Memory (ROM). The media is not written to directly by an e-beam but is fabricated with data imprinted onto the media by using patterned layers with different SE rates. Although two layers are to be expected as the most useful, several different layers of materials with different SE rates could be used.

In yet another alternate embodiment, a multi layer media can be employed where the bottom layer is a conductive metal that allows electron mobility so as to replenish data areas recently depleted by a read beam and to minimize local heating.

While each of these embodiments have been described as separate embodiments, properties of one embodiment may be found in other embodiments. For example, a multi-layer medium allowing for vaporization of a second layer to expose a third layer and remove a first layer may also include a bottom layer (either the third layer or a different layer) having electron mobility. Similarly, a read-only medium may be formed in most or all of the embodiments described, for example.

In some embodiments of a medium, the first layer can be any of a wide number of materials exhibiting a low secondary emission with the second layer a material with a higher secondary emission ratio, or vice versa. One embodiment of such a medium is composed of two layers deposited sequentially onto a substrate with a smooth surface. If a conductive under layer is used it can also function as a smoothing layer. Some experimentation suggests that the denser the material, the smoother that material tends to be.

During write mode the e-beam is focused to a small region of the media and has an electron energy (i.e. voltage) that corresponds to complete absorption of the beam in the top layer. The beam voltage and the top layer thickness are selected so that the beam heats only the top layer and vaporizes it, but leaves the second layer essentially unchanged. The write beam voltage is determined by the criteria of just complete absorption of the write beam electrons, and the necessary beam energy to achieve vaporization is selected by adjusting the beam current as a function of beam size and the chosen top layer material. The read voltage is selected to obtain a maximum value of δ for the higher SE ratio material (either top of bottom layer) during the read process, but with the current adjusted so that writing does not occur.

For example, if magnesium oxide (MgO) were the higher SE material a maximum value of δ will occur at 1500v, which is therefore the desired read voltage. For just complete absorption of the primary electrons of 1500v energy in the first layer a thickness of 2.23×10⁻⁵ gms/cm² is necessary, regardless of the top layer composition.

If the top layer is a plastic, for example PMMA, with a density of 1.2 gms/cm³, the desired thickness to just reach 100% beam absorption is (2.23×10⁻⁵)/1.2 cms. or 186 nanometers. Hence a 1500v primary beam will just be absorbed in the top layer when writing (minimizing the necessary write current) and will maximize the SE rate of the second layer on read. Different beam voltages can also be applied for the write and read processes. For material combinations where the top layer has a lower vaporization temperature than the lower layer some absorption of the beam energy in the lower layer may be acceptable, enabling the layer thickness and therefore recorded spot size to be reduced by 20%. The media substrate can be chosen without regard to its SE characteristics.

As shown below different materials possess different optimum thicknesses for 100% absorption as a function of their density and the impinging beam voltage. The absorption constants for 3 keV, 2 keV, and 1.5 keV electron beams are 5.3×10⁻⁵; 3.17×10⁻⁵; and 2.23×10⁻⁵; gms/cm², respectively.

The thickness of material that will just absorb 100% of the input beam energy is a function of material density with more dense materials providing thinner absorbing layers. As the top layer is ablated or vaporized to record a spot the minimum useful recorded spot size relates to the layer thickness, and is expected to be between half and one quarter of the layer thickness.

The layer thicknesses for 100% absorption for some selected materials are given in Table 1.

TABLE 1
Material thickness (nanometers) for 100% absorption of e-beam of given voltage.

| | | Material | | | | | |
|---|---|---|---|---|---|---|---|
| | | PMMA | Aluminum | Diamond | Titanium | Chromium | Tungsten |
| | | Density, gms/cc. | | | | | |
| | | 1.2 | 2.7 | 3.5 | 4.5 | 7.1 | 19.3 |
| Thickness | @ 3 keV | 442 | 196 | 151 | 118 | 74 | 27 |
| | @ 2 keV | 264 | 117 | 90 | 70 | 44 | 16 |
| | @ 1.5 keV | 186 | 82 | 64 | 50 | 31 | 12 |

From Table 1 it is clear that to record spot sizes in the low nanometer range a material of high density should be used in conjunction with a low beam voltage.

The voltage values in Table 1 are useful for recording systems where some passage of the beam through air or a window or both is desired. Operation in a vacuum allows lower beam voltages to be used, while still allowing 100% absorption in thinner media layers, leading to smaller recorded spot sizes. For example, a 500v beam is fully absorbed in a 7.5 nm thick layer of Chromium and potentially allows recording of 2 nm spots. The lower beam voltage also allows more electrons at a given beam energy thus improving write pulse repeatability.

Typical Write Energy Requirements, 2 keV Beam in a Chromium Layer.

In one exemplary embodiment an electron pulse writes to a thin Chromium layer covering an underlying magnesium oxide layer with an SE ratio of 20.

For a written mark of 15 nm diameter in a Chromium layer 44 nm thick the volume of material is: Volume (cm³)=p(7.5×10⁷)²×44×10−77.8×10⁻¹⁸ cm³
For a material density of 7.1 gms/cm³ the mass is m=5.5×10⁻¹⁷ gms.
For a specific heat of 4.6 Joule/cc·deg·K the Energy to raise the mass by 2650 degrees Kelvin is:
dH=5.5×10⁻¹⁷×1/4.6×2,650=3.2×10⁻¹⁵ Joule/spot.

Energy of vaporization=12 k J/cc=9.4×10⁻¹⁴ Joules/spot.

Total energy to vaporize a 15 nm spot=9.7×10⁻¹⁴ J.

This corresponds to only 600 electrons at 1 keV or 300 electrons at 2 keV.

Hence a write pulse consisting of 300 electrons at 2 keV each will vaporize a 15 m spot, leaving the underlying media exposed. If the lower layer has a secondary emission ratio (δ) of 20 and is read by a pulse of 200 electrons of 1.5 keV each, then an emission of 4,000 electrons results. If read in one nanosecond this produces a current of 640 nanoamps. With a preamplifier noise current of 3×10⁻¹² amps·√Hz., this provides a signal to noise ratio of −6.6, or 16 dB.

Typical Write Energy Requirements, 1.5 keV Beam in a Diamond Layer.

In another embodiment, the top layer is CVD (chemical vapor deposition) diamond that vaporizes forming carbon dioxide gas. No solid debris is created to contaminate the remainder of the disk surface or elsewhere. The second layer is magnesium oxide (MgO) in one embodiment. For a diamond density of 3.5 gms./cc. and a primary beam energy of 1,500 volts a CVD diamond thickness of 64 nm is required for nominally 100% beam absorption. The volume vaporized per 15 nm mark is 1.13×10⁻¹⁷ cm³

For a material density of 3.5 gms/cm³ the mass is m=4.0×10⁻¹⁷ gms.

For a diamond vaporization energy of 60 kJ/gm, the vaporization energy per 15 nm spot=2.4×10⁻¹² J. This corresponds to a pulse of 10,000 electrons at 1.5 keV each. With diamond as one layer with an SE ratio of 2.8, the other layer could be of either a lower or a higher SE ratio.

Other Materials

In yet another embodiment the recording media is a phase change material that undergoes a change of state on being heated to an appropriate temperature. For one known material available from MicroContinuum Corp. of Cambridge, Mass. (formerly from Polaroid Corp.) the energy to change phase of a 20 nm diameter spot on a 40 nm thick layer is 2.3×10⁻¹³ J., a write energy between those of the first two examples and representing 1,000 electrons at 1,500 eV each. Materials such as these are also available from Imation Corp. of Minneapolis, Minn. (formerly from Eastman Kodak). Typically, the exact formulations of such materials are not made available, but such materials often include a mixture of Indium, Tin and Antimony, for example. Some examples of phase change media offer a reversible process based on thermal cycling. In some embodiments, it may be preferable to use a phase change material which changes phase at high temperatures.

A selection of materials with their secondary emission ratio and other relevant parameters is given in Table 2. The literature has incomplete data on some materials.

TABLE 2
Values of δ and the corresponding primary voltage for selected materiais.

| | | | melt | | Density, |
|---|---|---|---|---|---|
| Material | δ | Voltage | T ° C. | Vap. T ° C. | gm/cm³. |
| Phase Chg Mtl | 6 | | 300 | 2,000 | 7.0 |
| Diamond | 2.8 | 750 | — | 3,827 | 3.51 |
| Au | 1.4 | 800 | 1,063 | 2,808 | 19 |
| NaI xtl | 19 | 1,300 | | | |
| NaI layer | 5.5 | | | | |
| KCl xtl. | 12 | 1,600 | 771 | 1,427 | 2.0 |
| KCl Layer | 7.5 | | | | |
| KBr xtl | 14 | 1,800 | | | |
| NaCl xtl. | 14 | 1,200 | 800 | 1,457 | 2.16 |
| NaCl Layer | 6.8 | 600 | | | |
| MgO xtl. | 25 | 1,500 | 2,852 | — | 3.65 |
| MgO Layer | 4-15 | 1,500 | | | |
| GAP + Cs. | 120¹ | 2,500 | 1,350 | | |
| MgF₂ | 4 | 400 | 1,252 | 1,252 | 3.15 |
| LiF | 5.6 | 700 | | | |
| Al₂O₃ | 2-9 | | 2,323 | | |
| R_{b}S_{b} | 7.1 | 450 | | | |
| Si | 1.1 | 250 | 1,412 | 3,267 | 2.4 |
| Ti | 0.9 | 280 | 1,760 | 3,289 | 4.5 |
| M_{g} | 0.95 | 300 | 922 | 1,363 | |
| W | 1.4 | 650 | 3,653 | 5,828 | 19.3 |

Read Only Memory

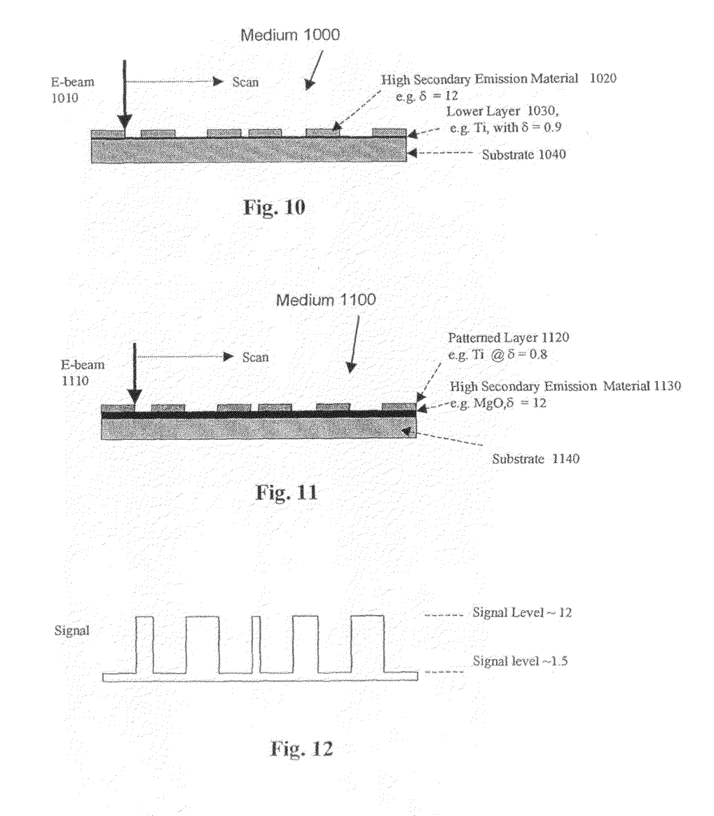

In one embodiment a memory device is created using micro-lithography or Nano Imprint Lithography (NIL) to record nanoscale data patterns. This can be achieved in two ways. By coating a layer of material with high secondary emission electron rates onto a substrate with much lower emission rates. Where the material is not accessible, or absent due to either its removal or lack of deposition due to a mask, the secondary emission is that of the substrate, or of a first layer coated onto the substrate below the recording layer. For example, if a thin layer of MgO were deposited through a nanoscale imprint mask onto a glass substrate first coated with a titanium layer as in FIG. 10, e-beam scanning of the structure will provide a substantial signal on-off ratio. At the peak voltage for MgO, 1500v, Ti produces less than 0.9 secondary electrons per primary electron, while MgO gives about 13, giving a signal on-off ratio of approximately 15:1.

An alternative approach is to coat the substrate with a uniform MgO layer and then deposit a patterned blocking layer such as titanium on top of the MgO. The titanium is patterned with data either by deposition through a mask or by resist removal.

### Additional Examples and Embodiments

Many other variations of the proposed real-time recording media exist, such as one employing a hydrogenated diamond like layer as the higher SE ratio material. Materials of this nature have shown SE ratios in excess of 75. The improved SE ratio arises from a reduction in the work function of the sensitive layer surface by the hydrogenation process. Other means of reducing the effective work function of the surface may be employed such as applying a bias voltage.

As mentioned previously, FIG. 10 illustrates an embodiment of a read-only medium for use in a storage device. Medium 1000 includes a substrate 1040, upon which a lower layer 1030 and an upper layer 1020 are formed. As illustrated, upper layer 1020 has a high secondary emission, such as about 12 for example, and lower layer 1030 has a low secondary emission, such as about 0.9 for example. As a result, when e-beam 1010 scans across medium 1000, a signal may be produced, where the detected current in the form of secondary emission electrons varies depending on which material (layer 1020 or layer 1030) is under scan at the moment.

Similarly, FIG. 11 illustrates an alternate embodiment of a read-only medium for use in a storage device. Substrate 1140 has formed thereon lower layer 1130 (high secondary emission material), and upper layer 1120 (low secondary emission material). When e-beam 1110 scans across medium 1100 (or medium 1100 moves underneath e-beam 1110), a current waveform such as that illustrated in FIG. 12 may be detected, as a result of secondary emission of electrons from upper layer 1120 and lower layer 1130.

For both FIG. 11 and FIG. 10, the patterns of the upper layers 1020 and 1120 are formed during fabrication of the medium, thus providing a read-only form of media. For example, the media of FIG. 10 may be formed by depositing lower layer 1030 on substrate 1040, then forming upper layer 1020 on lower layer 1030, either through a writing process, or through some form of resist process for example. Similar forms of media discussed below may be used for write-once and read-write media. In almost all media, modifications may be made (the media may be written), with sufficient effort. However, read-only media is intended to only be read.

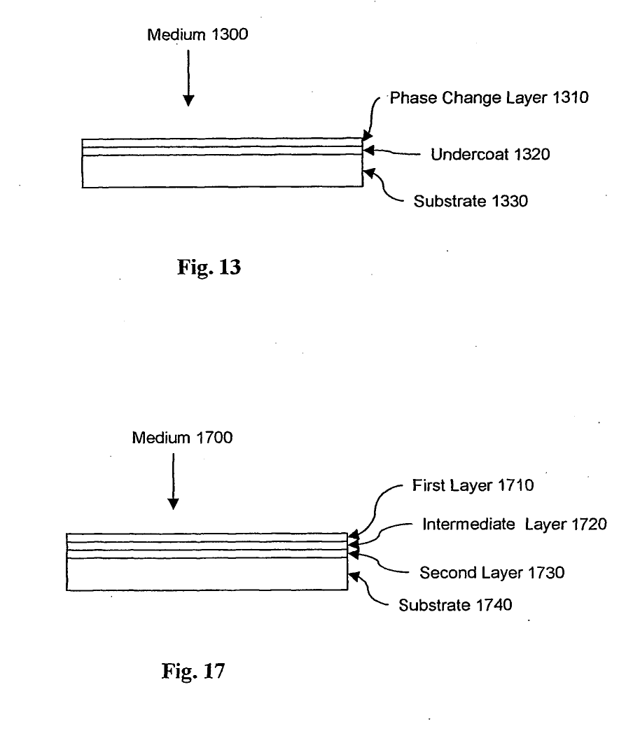

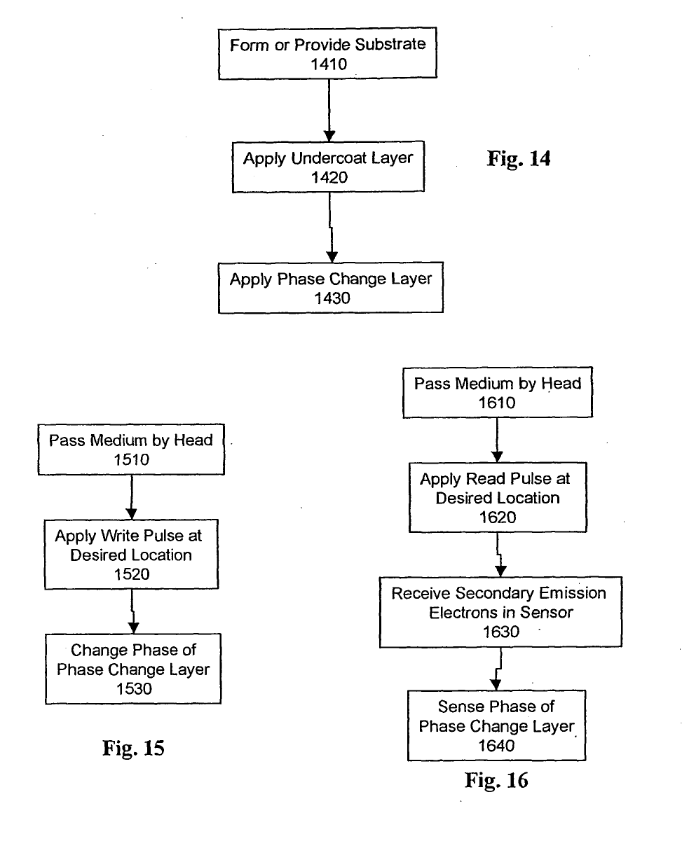

Phase change media, for example, may be formed in a similar manner to read-only media, while allowing for read-write performance. FIG. 13 illustrates an embodiment of a phase change medium for use in a storage device. FIG. 14 illustrates a method of making a phase change medium for use in a storage device. While the medium of FIG. 13 may be formed in a variety of ways, and the process of FIG. 14 may produce a variety of different types of media, the two figures may be understood collectively.

At module 1410, a substrate such as substrate 1330 is provided for medium 1300. Alternatively, substrate 1330 may be formed as part of the process, depending on commercial availability and manufacturing tolerances. Substrate 1330 may be various materials, such as plastic, glass or metal for example. At module 1420, an undercoat layer 1320 is formed, such as through deposition or spin-on methods. Undercoat layer 1320 may be a metal, such as Aluminum for example, a plastic, such as PMMA for example, or some other suitable material. At module 1430, the phase change layer 1310 is applied to undercoat 1320, such as through chemical vapor deposition for example. The phase change layer 1310 may be formed of a material which has a relatively low melting point and a relatively high vaporization point, for example. Medium 1300 may include additional layers, and may be formed through additional modules, such as a hydrogen implantation module for example.

Once formed, phase change material must be written to and read from to be useful. FIG. 15 illustrates a method of writing a phase change medium for use in a storage device. At module 1510, the medium (such as medium 1300) is passed under or by a write head (which may be a dedicated write head or a read-write head for example). Passing by the write head may be a result of rotation or movement of the medium, the head, or both.

At module 1520, a write pulse is applied at the desired write location. Preferably, such a pulse heats up the medium enough to change its phase within a small area, defined as the spot-size. In one embodiment, the phase-change material has both a crystalline and an amorphous phase, both of which are essentially stable at operating temperatures. Upon heating, the material may be rapidly cooled, potentially causing an amorphous phase to form, or slowly cooled, potentially causing a crystalline phase to form. Thus, at module 1530, the phase of the phase change layer is changed.

Having written the media, it must also be read to be useful. FIG. 16 illustrates a method of reading a phase change medium for use in a storage device. At module 1610, the medium is passed by a read head (which may be a dedicated head or a read-write head for example). At module 1620, a read pulse is applied at the desired location. At module 1630, secondary emission electrons are received in an associated sensor of the read head. At module 1640, the phase of the phase change layer is sensed. For example, in some embodiments, the secondary electron emission for crystalline and amorphous phases of a phase change material may have a 10:1 or greater ratio, allowing for relatively simple sensing.

Methods of FIGS. 15 and 16 may include additional modules, such as tracking and adjusting position, or locating a desired portion of a medium for example. Moreover, methods in general may include additional or varied modules beyond those mentioned specifically, within the broader spirit and scope of various embodiments. Similarly, apparatuses or systems may include additional components beyond those mentioned specifically, and may include rearranged components in some cases, as well.

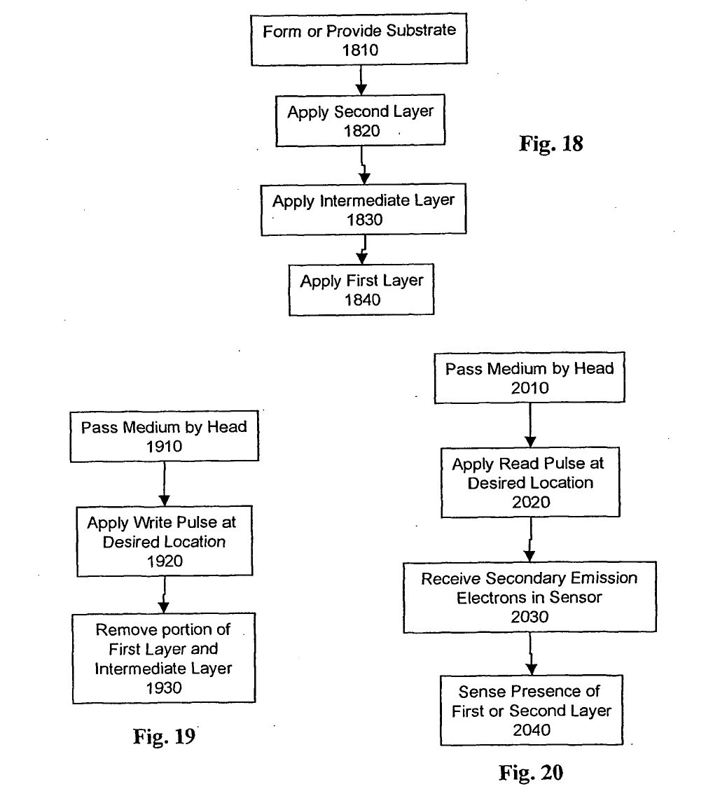

Alternatively, write-once media may be used. FIG. 17 illustrates an embodiment of a write-once medium for use in a storage device. FIG. 18 illustrates a method of making a write-once medium for use in a storage device. While the medium of FIG. 17 may be formed in a variety of ways, and the process of FIG. 18 may produce a variety of different types of media, the two figures may be understood in conjunction.

At module 1810, a substrate such as substrate 1740 is provided for medium 1700. Alternatively, substrate 1740 may be formed as part of the process. Substrate 1740 may be various materials, such as plastic, glass or metal for example. At module 1820, a second layer 1730 is formed, such as through deposition or spin-on methods. Second layer 1730 may be a metal, such as Aluminum for example, a plastic, such as PMMA for example, or some other suitable material, with either a relatively high secondary emission ratio or a relatively low secondary emission ratio. At module 1830, the intermediate layer 1720 is applied to second layer 1730, such as through chemical vapor deposition for example. Intermediate layer 1720 may be a layer meant to be ablated or vaporized but not sensed, for example. At module 1840, the first layer 1710 is applied to intermediate layer 1720, such as through chemical vapor deposition for example. The first layer 1710 may be formed of a material which has a contrasting secondary emission ratio from that of second layer 1730, for example. Medium 1700 may include additional layers, and may be formed through additional modules, such as a hydrogen implantation module for example, or may omit the intermediate layer 1720 for example.

Once formed, write-once media must be written to and read from to be useful. FIG. 19 illustrates a method of writing a write-once medium for use in a storage device. At module 1910, the medium (such as medium 1700) is passed under or by a write head (which may be a dedicated write head or a read-write head for example). At module 1920, a write pulse is applied at the desired write location. Preferably, such a pulse heats up the medium enough to evaporate or ablate the first layer and intermediate layer within a small area, defined as the spot-size. Upon heating, the material may effectively be removed, either as a result of the rapidity of heating, or the force of corresponding vaporization for example. Therefore, at module 1930, the material of the first layer and the intermediate layer is removed. In some embodiments, vaporization, with its lack of residual material, may be preferable.

Once written (or before writing), the medium may also be read. FIG. 20 illustrates a method of reading a write-once medium for use in a storage device. At module 2010, the medium is passed by a read head (which may be a dedicated head or a read-write head for example). At module 2020, a read pulse is applied at the desired location. At module 2030, secondary emission electrons are received in a detector or sensor of the read head. At module 2040, the layer (either the first or second layer for example) is sensed. For example, depending on design choices for the medium, the secondary electron emission for the first and second layers of a write-once medium may have a 15:1 or greater ratio, allowing for relatively simple sensing. Under other circumstances, a ratio as small as 2:1 may be sufficient.

In the foregoing detailed description, the method and apparatus of the present invention has been described with reference to specific exemplary embodiments thereof. It will, however, be evident that various modifications and changes may be made thereto without departing from the broader spirit and scope of the present invention. In particular, the separate blocks of the various block diagrams represent functional blocks of methods or apparatuses and are not necessarily indicative of physical or logical separations or of an order of operation inherent in the spirit and scope of the present invention. For example, the various blocks of an apparatus or system may be integrated into components, or may be subdivided into components. Similarly, the blocks of a method represent portions of the method that, in some embodiments, may be reordered or may be organized in parallel rather than in a linear or step-wise fashion. The present specification and figures are accordingly to be regarded as illustrative rather than restrictive.

1.-6. (canceled)

7. A method, comprising:
receiving a substrate; and
depositing on the substrate a first layer of material having a first secondary emission ratio.

8. The method of claim 7, wherein:
the first secondary emission ratio is associated with a first phase of the first layer, and the first layer has a second phase with an associated second secondary emission ratio.

9. The method of claim 7, further comprising:
depositing on the first layer a second layer having a second secondary emission ratio.

10. The method of claim 9, wherein:
the second secondary emission ratio is greater than the first secondary emission ratio by a factor of at least 10.

11.-15. (canceled)

16. A method, comprising:
receiving electrons at a spot of a first layer of a medium, the first layer disposed above a second layer, the first layer having a first secondary emission ratio, the second layer having a second secondary emission ratio, the first secondary emission ratio differing from the second secondary emission ratio; and
removing a portion of the first layer responsive to receiving the electrons, the portion aligned with the spot.

17. The method of claim 16, wherein:
the portion is ablated during removing the portion of the first layer.

18. The method of claim 16, wherein:
the portion is vaporized during removing the portion of the first layer.

19. The method of claim 16, wherein:
the electrons are further received in an intermediate layer disposed between the first layer and the second layer;
and further comprising:
ablating the intermediate layer in alignment with the spot, the ablating the intermediate layer also removing the first layer.

20. A method, comprising:
projecting electrons from a carbon nanotube at a spot of a first layer of a medium, the first layer disposed above a second layer, the first layer having a first secondary emission ratio, the second layer having a second secondary emission ratio, the first secondary emission ratio differing from the second secondary emission ratio, the number and energy of electrons projected based on an expected amount of energy to remove a portion of the first layer, the portion of the first layer aligned with the spot; and
removing a portion of the first layer responsive to receiving the electrons.

21. The method of claim 20, wherein:
the portion of the first layer is ablated during removing the portion of the first layer.

22. The method of claim 20, wherein:
the portion of the first layer is vaporized during removing the portion of the first layer.

23. The method of claim 20, wherein:
the electrons are further received in an intermediate layer disposed between the first layer and the second layer, the expected amount of energy to remove the first layer includes energy to ablate the intermediate layer;
and further comprising:
ablating the intermediate layer in alignment with the spot, the ablating the intermediate layer also removing the first layer.

24. A method, comprising:
projecting electrons from a carbon nanotube at a spot of a phase change material having a first phase and a second phase, the first phase having associated therewith a first secondary emission ratio, the second phase having associated therewith a second secondary emission ratio; and
absorbing the electrons within a portion of the phase change material, the portion aligned with the spot, the portion in the first phase prior to absorbing the electrons;
changing the portion of the phase change material from the first phase to the second phase responsive to absorbing the electrons.

25. The method of claim 24, further comprising:
cooling the portion of the phase change material quickly after absorbing the electrons.

26. The method of claim 24, further comprising:
cooling the portion of the phase change material slowly after absorbing the electrons.

27.-28. (canceled)
